(19) United States
(12) Patent Application Publication
(10) Pub. No.: US 2020/0311928 A1
(43) Pub. Date: Oct. 1, 2020

(54) MEDICAL IMAGE PROCESSING APPARATUS, MEDICAL IMAGE PROCESSING METHOD, AND MEDICAL IMAGE PROCESSING SYSTEM

(71) Applicant: ZIOSOFT, INC., Tokyo (JP)

(72) Inventor: Takuro HAYASHIDA, Tokyo (JP)

(21) Appl. No.: 16/832,303

(22) Filed: Mar. 27, 2020

(30) Foreign Application Priority Data
Mar. 28, 2019 (JP) 2019-064197

(51) Int. Cl. G06T 7/00; A61B 34/10; A61B 34/37; A61B 34/00

(52) U.S. Cl. CPC G06T 7/0012; A61B 34/10; A61B 34/37; G06T 2207/30024; G06T 2200/04; A61B 2034/102; G06T 2207/10081; A61B 34/25

(57) ABSTRACT

A medical image processing apparatus includes: a display unit; and circuitry configured to: acquire volume data of a subject; set a target to be placed inside the subject in 3D data based on the volume data; and visualize the 3D data to display an image and information on the display unit. In the image, an epithelial tissue of the subject is not displayed, is transparent, or is semi-transparent. The information represents a port placement range where a port through which a surgical instrument reaching the target is inserted is placeable on the epithelial tissue.

### TECHNICAL FIELD

The present disclosure relates to a medical image processing apparatus, a medical image processing method, and a medical image processing system.

### BACKGROUND ART

In the related art, as one of surgical procedures, single incisional laparoscopic surgery (SILS) is known. In SILS, in order to perform a medical procedure on a target such as affected part, a surgical instrument is inserted through a single port (refer to U.S. Pat. No. 8,517,933B2).

The present disclosure provides a medical image processing apparatus enables the user to recognize a range where a port which a surgical instruments pass though is placeable in a subject and reducing burden on an operator and the subject, a medical image processing method, and a medical image processing system.

### SUMMARY

A medical image processing apparatus of one aspect of the present disclosure includes: a display unit; and circuitry configured to: acquire volume data of a subject; set a target to be placed inside the subject in 3D data based on the volume data; and visualize the 3D data to display an image and information on the display unit. In the image, an epithelial tissue of the subject is not displayed, is transparent, or is semi-transparent. The information represents a port placement range where a port through which a surgical instrument reaching the target is inserted is placeable on the epithelial tissue.

According to the present disclosure, a range where a port through which a surgical instrument passes is placeable on a subject can be recognized, and burden on an operator and the subject can be reduced.

### BRIEF DESCRIPTION OF DRAWINGS

FIG. 1 is a block diagram illustrating a hardware configuration example of a medical image processing apparatus according to a first embodiment;

FIG. 2 is a block diagram illustrating a functional configuration example of the medical image processing apparatus;

FIG. 3 is a diagram illustrating a first display example of port range information.

FIG. 4 is a diagram illustrating a second display example of the port range information;

FIG. 5 is a diagram illustrating a third display example of the port range information;

FIG. 6 is a diagram illustrating a fourth display example of the port range information;

FIG. 7 is a diagram illustrating a fifth display example of the port range information;

FIG. 8 is a diagram illustrating a sixth display example of the port range information;

FIG. 9 is a diagram illustrating a seventh display example of the port range information; and

FIG. 10 is a flowchart illustrating an operation example of the medical image processing apparatus.

### DESCRIPTION OF EMBODIMENTS

Hereinafter, an embodiment of the present disclosure will be described with reference to the drawings.

A medical image processing apparatus of the present embodiments includes: a display unit; and circuitry configured to: acquire volume data of a subject; set a target to be placed inside the subject in 3D data based on the volume data; and visualize the 3D data to display an image and information on the display unit. In the image, an epithelial tissue of the subject is not displayed, is transparent, or is semi-transparent. The information represents a port placement range where a port through which a surgical instrument reaching the target is inserted is placeable on the epithelial tissue.

Accordingly, a user can recognize the range where the port through which the surgical instrument is inserted is placeable on the subject, and burden on an operator and the subject can be reduced. By placing (piercing) the port in the port placement range, the user can improve safety of a treatment on the target. The user can conceive a required treatment in consideration of the information regarding the port placement range in the preoperative simulation or the intraoperative navigation. In addition, by not displaying the epithelial tissue such that the epithelial tissue is transparent or semi-transparent, the user can easily grasp the port placement range, the placement of the shaft of the forceps from the port to the target, the insertion path of the forceps, and the target position intuitively at the same time while checking the target and the surrounding tissue.

### Circumstances on How Aspect of Present Disclosure is Obtained

In SILS, in order to keep airtightness in a subject, a port airtightness keeping instrument is used. In SILS, the port airtightness keeping instrument is designed to be relatively thick, and it is difficult to obliquely tilt the port airtightness keeping instrument on a body surface. Therefore, in SILS, a place where a port is placeable is limited. In addition, for example, burden on the body surface (skin) of the subject into which a surgical instrument is inserted is likely to increase. In this case, when a target is designated, it is preferable that a range (port placement range) where a port through which a surgical instrument is inserted is placeable can be recognized because the port can be easily placed and burden on an operator and the subject can be reduced.

In the following embodiment, a medical image processing apparatus capable of recognizing a range where a port through which a surgical instrument passes is placeable on a subject and reducing burden on an operator and the subject, a medical image processing method, and a medical image processing system will be described.

### First Embodiment

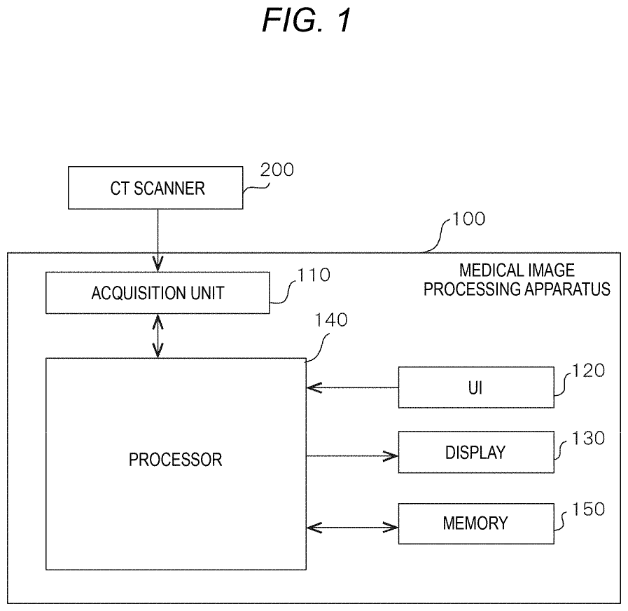

FIG. 1 is a block diagram illustrating a configuration example of a medical image processing apparatus 100 according to a first embodiment. The medical image processing apparatus 100 includes an acquisition unit 110, a UI 120, a display 130, a processor 140, and a memory 150. The medical image processing apparatus 100 supports surgery by a manual operation of an operator (also referred to as manual surgery) or robotic surgery through image processing.

A CT scanner 200 is connected to the medical image processing apparatus 100. The medical image processing apparatus 100 acquires volume data from the CT scanner 200 and processes the acquired volume data. The medical image processing apparatus 100 may be configured with a PC and software installed in the PC.

The CT scanner 200 irradiates a subject with X-rays and acquires images (CT images) using a difference in X-ray absorption depending on tissues in the body. The subject may include an organism, a human body, an animal, and the like. The CT scanner 200 generates volume data including information regarding any portion inside the subject. The CT scanner 200 transmits the volume data as the CT image to the medical image processing apparatus 100 via a wired circuit or a wireless circuit. In order to acquire the CT image, scanning conditions regarding CT scanning or contrast enhancement conditions regarding contrast medium administration may be considered.

The acquisition unit 110 inside the medical image processing apparatus 100 includes a communication port, an external device connection port, or a connection port to an embedded device and acquires volume data obtained by the CT scanner 200. The acquired volume data may be transmitted immediately to the processor 140 for various processes, or may be stored in the memory 150 first and then transmitted to the processor 140 as necessary for various processes. In addition, the volume data may be acquired via a recording medium. The volume data may be acquired in the form of intermediate data, compressed data, or sinogram. The volume data may be acquired from information from a sensor device attached to the medical image processing apparatus 100. This way, the acquisition unit 110 has a function of acquiring various data such as volume data.

The UI 120 may include, for example, a touch panel, a pointing device, a keyboard, or a microphone. The UI 120 receives any input manipulation from a user of the medical image processing apparatus 100. The user may include an operator, a medical doctor, a nurse, a radiology technician, a student, or the like.

The UI 120 receives various operations. For example, the UI 120 receives operations such as designation of a region of interest (ROI) or setting of luminance conditions in volume data or an image based on the volume data (for example, a three-dimensional image or a two-dimensional image to be described below). The region of interest may include regions of various tissues (for example, blood vessels, the bronchi, organs, bones, and the brain). The tissues may include lesion tissue, normal tissue, tumor tissue, and the like.

The display 130 may include, for example, an LCD, and displays various information. Various information may include a three-dimensional image or a two-dimensional image obtained from volume data. The three-dimensional image may include a volume rendering image, a surface rendering image, a virtual endoscopic image, a virtual ultrasound image, a CPR image, and the like. The volume rendering image may include a RaySum image, an MIP image, an MinIP image, an average value image, or a raycast image. The two-dimensional image may include an axial image, a sagittal image, a coronal image, an MPR image, and the like.

The memory 150 includes various primary storage devices such as ROM or RAM. The memory 150 may include a secondary storage device such as an HDD or an SSD. The memory 150 may include a tertiary storage device such as a USB memory or an SD card. The memory 150 stores various information and programs. The various information may include volume data acquired by the acquisition unit 110, an image generated by the processor 140, setting information set by the processor 140, and various programs. The memory 150 is an example of a non-transitory recording medium in which programs are recorded.

The processor 140 may include a CPU, a DSP, or a GPU. The processor 140 functions as a processing unit 160 performing various kinds of processing and controls by executing a medical image processing program stored in the memory 150.

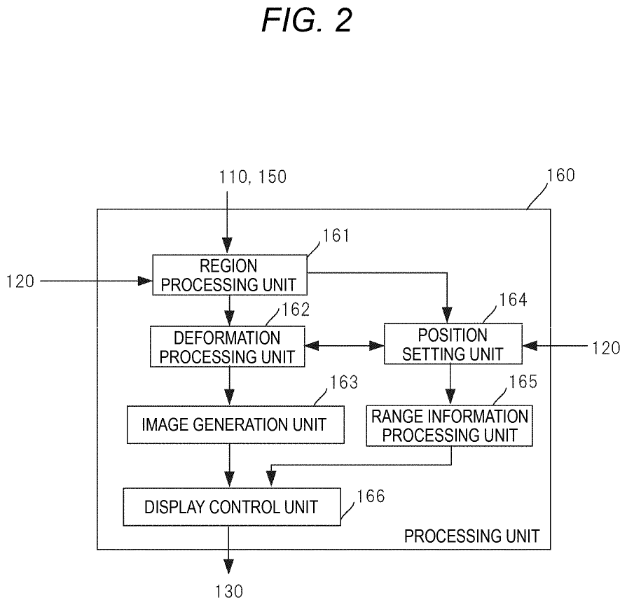

FIG. 2 is a block diagram illustrating a functional configuration example of the processing unit 160. The processing unit 160 includes a region processing unit 161, a deformation processing unit 162, an image generation unit 163, a position setting unit 164, a range information processing unit 165, and a display control unit 166. The respective units included in the processing unit 160 may be realized as different functions using one hardware device or may be realized as different functions using a plurality of hardware devices. The respective units included in the processing unit 160 may be realized using exclusive hardware components.

The region processing unit 161 acquires volume data of a subject, for example, via the acquisition unit 110. The region processing unit 161 extracts any region in the volume data. The region processing unit 161 may extract a ROI by automatically designating the ROI based on pixel values of the volume data, for example. The region processing unit 161 may extract a ROI by manually designating the ROI via, for example, the UI 120. The ROI may include regions of organs, bones, blood vessels, affected part (for example, lesion tissue or tumor tissue), or the like.

The deformation processing unit 162 may perform a process relating to deformation in the subject as a surgery target. For example, the deformation processing unit 162 may execute a pneumoperitoneum simulation of virtually performing pneumoperitoneum on the subject. A specific method of the pneumoperitoneum simulation may be a well-known method, for example, a method described in Takayuki Kitasaka, Kensaku Mori, Yuichiro Hayashi, Yasuhito Suenaga, Makoto Hashizume, and Junichiro Toriwaki, “Virtual Pneumoperitoneum for Generating Virtual Laparoscopic Views Based on Volumetric Deformation”, MICCAI (https://link.springer.com/book/10.1007/b100270, Medical Image Computing and Computer-Assisted Intervention), 2004, P559-P567. That is, the deformation processing unit 162 generates volume data of a virtual pneumoperitoneum state by performing the pneumoperitoneum simulation based the volume data of a non-pneumoperitoneum state. Through the pneumoperitoneum simulation, the user simulates a state where pneumoperitoneum is performed on the subject without actually performing pneumoperitoneum on the subject, and can observe a state where pneumoperitoneum is virtually performed. Among pneumoperitoneum states, a state of pneumoperitoneum estimated by the pneumoperitoneum simulation will be referred to as “virtual pneumoperitoneum state”, and a state where pneumoperitoneum is actually performed will also be referred to as “actual pneumoperitoneum state”.

The pneumoperitoneum simulation may be a large deformation simulation using a finite element method. In this case, the deformation processing unit 162 may segment a body surface including subcutaneous fat of the subject and an abdominal organ of the subject. The deformation processing unit 162 may model the body surface as a two-layer finite element including skin and body fat and may model the abdominal organ as a finite element. For example, the deformation processing unit 162 may optionally segment a lung and a bone to be added to the model. In addition, the deformation processing unit 162 may provide a gas region between the body surface and the abdominal organ and may extend (expand) the gas region (pneumoperitoneum space) by virtual gas injection.

The deformation processing unit 162 may virtually deform a target such as an organ or a disease in the subject. The deformation processing unit 162 may simulate a state where an organ is pulled, pressed, or dissected by forceps. In addition, the deformation processing unit 162 may simulate, for example, movement of an organ by a postural change. In this case, an elastic force applied to a contact point of an organ or a disease, stiffness of an organ or a disease, or other physical properties may also be considered.

The image generation unit 163 generates various images. The image generation unit 163 generates a three-dimensional image or a two-dimensional image based on at least a part of the acquired volume data (for example, the extracted region in the volume data). The image generation unit 163 may generate a three-dimensional image or a two-dimensional image based on volume data deformed by the deformation processing unit 162 (for example, volume data in the virtual pneumoperitoneum state or volume data deformed according to movement of a surgical instrument (for example, forceps, a camera, or other surgical instruments)). The forceps may include, for example, a bipolar grasper, a maryland dissector, or an acusector.

The position setting unit 164 sets a target that is reached by a surgical instrument in the subject. The target may be a point or a region in the subject. The target may be set in the volume data of the non-pneumoperitoneum state (the volume data before pneumoperitoneum) or may be set in the volume data of the virtual pneumoperitoneum state (pneumoperitoneum volume data). The target may be the same as the ROI or may be a point or a region (for example, affected part) that is included in the ROI, and to which the user particularly wants to pay attention. In addition, the target may be the entire organ including a point or a region to which the user wants to pay attention or may be a part (for example, a section or a dominant blood vessel) of the organ including a point or a region. A method of setting the target may be the same as the method of designating the ROI and may be manually or automatically set.

The range information processing unit 165 derives (for example, calculates) a port placement range on the body surface of the subject based on the position of the target set in the subject. The port placement range has various shapes in a three-dimensional space. The range information processing unit 165 generates port range information including the port placement range.

The range information processing unit 165 acquires a port placement condition for placing the port. The port placement condition may be read from the memory 150 or may be acquired from an external server via the acquisition unit 110. The range information processing unit 165 determines whether or not the port placement condition is satisfied. For example, the range information processing unit 165 may determine whether or not the port placement condition is satisfied for each voxel in the subject. A voxel that satisfies the port placement condition is included in the port placement range, and a voxel that does not satisfy the port placement condition is excluded from the port placement range.

The port placement condition may include a condition that interference with an obstacle is avoidable. In this case, the range information processing unit 165 may calculate the port placement range in consideration of an obstacle that may obstruct the placement of the port on the subject. For example, the range information processing unit 165 may calculate the port placement range that passes through the target but does not pass through the position of the obstacle. The obstacle may include a tissue (for example, a blood vessel or an easily damaged organ) that needs to be careful when handled or a tissue (for example a bone) that has difficulty when the port is placed to physically overlap the tissue. The easily damaged organ may include an organ that may be damaged when directly stabbed, compressed, or pinched by forceps. The range information processing unit 165 may acquire information regarding characteristics of the obstacle (for example, properties of the obstacle or a range of the subject where the obstacle is present) such that the port placement range is calculated based on the information regarding the characteristics of the obstacle.

The port placement condition may include a condition that interference with other forceps is avoidable. In this case, the range information processing unit 165 may acquire information regarding a movable range (apparatus movable range) of the forceps reaching the set target. The apparatus movable range may be determined depending on the position of the set target or the characteristics of the forceps (for example, the size or the bending state of the forceps). Regarding the port placement range, information regarding a movable range (other movable range) of forceps other than the forceps reaching the set target (for example, forceps other than the forceps as a working target) may be acquired. The other movable range may be determined depending on the characteristics of the other forceps (for example, at least one of the use, the size, or the bending state of the other forceps). The range information processing unit 165 may calculate the port placement range in consideration of the interference with the other forceps other than the forceps reaching the set target. For example, the range information processing unit 165 may calculate the port placement range that passes through the target but does not pass through the movable range of the other forceps. For example, the information regarding the apparatus movable range and the other movable range may be stored in the memory 150 and read from the memory 150 or may be acquired from an external server via the acquisition unit 110.

The port placement condition may include an angle condition. This angle condition may include a condition that, when the port is set in the port placement range, an angle between the forceps to be inserted into the port at an estimated position of the port and the body surface of the subject is in a predetermined range where burden on the subject is allowed. In this case, the predetermined range may be, for example, a predetermined range in an angle range of more than 10° and less than 170°. In this case, 10° is the lower limit of the angle, and 170° is the upper limit of the angle. In this case, the maximum value of the angle is 180°. In addition, the maximum value of the angle may be 90° in consideration of symmetry of the angle between the forceps and the body surface. In this case, the angle is 10° to 90°. In this case, regarding the display of the angle, 10° or more may be displayed, or only the 10° side (lower limit side) may be displayed without displaying the 90° side.

In addition, for example, the port placement condition may include a condition that the forceps can pass through the target and the port (the estimated position of the port in the port placement range) with reference to characteristic information of the forceps. In this case, the range information processing unit 165 may acquire the characteristic information of the forceps in consideration of whether or not the forceps are linear or curved. This characteristic information may be acquired from an external server via the acquisition unit 110 or may be stored in the memory 150 to be read and acquired from the memory 150. The characteristic information of the forceps may include information on a bending method of the forceps. For example, when a shaft portion of the forceps has flexibility, the characteristic information may include information regarding the degree of flexibility. For example, when the shaft portion of the forceps does not have flexibility and is linearly foldable at one or more positions, the characteristic information may include information on the folding positions and folding angles (for example, curved forceps). That is, the forceps may be bent using a given method, and the angle of a distal end portion thereof is adjustable. The range information processing unit 165 may acquire characteristic information of other surgical instruments in addition to the forceps. The port placement range may be calculated to generate the port range information based on the characteristic information of the surgical instruments.

The deformation processing unit 162 may press the forceps against an organ or a disease to simulate deformation of the organ or the disease based on the characteristic information of the forceps.

The position setting unit 164 may set (plan) positions (port positions) of one or more ports to be placed (pierced) on the body surface of the subject in the derived port placement range. The position setting unit 164 may set the port positions in the volume data of the non-pneumoperitoneum state. In addition, the position setting unit 164 may set the port positions in the volume data of the virtual pneumoperitoneum state. In addition, the position setting unit 164 may set the port positions depending on a surgical procedure.

In addition, when robotic surgery is performed, the position setting unit 164 may set the port positions according to kinematic information of a surgical instrument (for example, an end effector or a robot arm corresponding to the forceps) for performing the robotic surgery. The kinematic information may include shape information regarding the shape of a surgical instrument or operation information regarding the operation of a surgical instrument. The port positions may be stored in the memory 150 or may be acquired from an external server via the acquisition unit 110. The port positions may be designated by the user via the UI 120.

The display control unit 166 causes the display 130 to display various data, information, or images. The images include an image (for example, the rendering image) generated by the image generation unit 163. The display control unit 166 may display the port range information including the port placement range to overlap the rendering image. In addition, the display control unit 166 may adjust the luminance of the rendering image. For example, the adjustment of the luminance may include adjustment of at least one of a window width (WW) and a window level (WL). Here, when a converted value of WW/WL is less than or equal to a predetermined value, the display may be transparent. The adjustment of the luminance may be performed, for example, using a look up table (LUT) in which a RGBA value corresponding to a voxel value is set.

Next, the surgical procedure that is assumed in the embodiment will be described.

The port range information may be displayed, for example, in SILS. SILS is one laparoscopic surgery, in which one port (hole) is placed on the abdomen of the subject, and a plurality of forceps are inserted through one port. The port for SILS is placed, for example, on the navel. As a result, postoperative trace is inconspicuous, and thus sense of esthetics is excellent, which is preferable in appearance. SILS is applicable to, for example, gynecologic surgery, cholecystectomy, appendectomy, and kidney surgery. In addition to the single port, an auxiliary port may also be placed. Even in this case, SILS may be adopted.

In natural orifice transluminal endoscopic surgery (NOTES), the port may be placed on the lumen. NOTES includes transanal total mesorectal excision (TaTME). In NOTES, when the lumen is pierced such that an endoscope is extended, the port is placed on the luminal surface instead of the body surface. Accordingly, the port placement range on the luminal surface may derived and displayed. In any of SILS, NOTES, or TaTME, either manual surgery or robotic surgery may be performed. In TaTME, for example, surgical instruments approaches affected part simultaneously in two directions from the abdominal cavity side and the rectum side. In addition, any one of laparoscopic surgery or minimally invasive robotic surgery may be performed using a plurality of forceps.

Next, a display example of the port range information will be described.

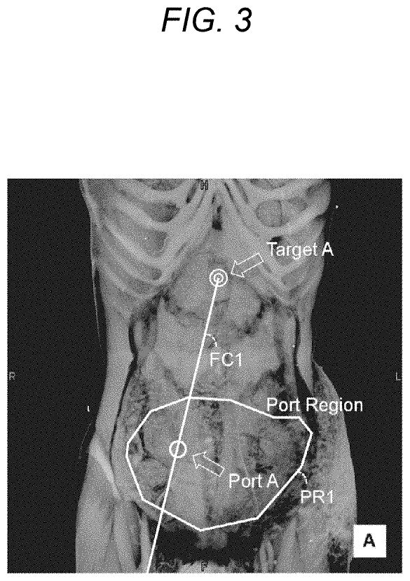

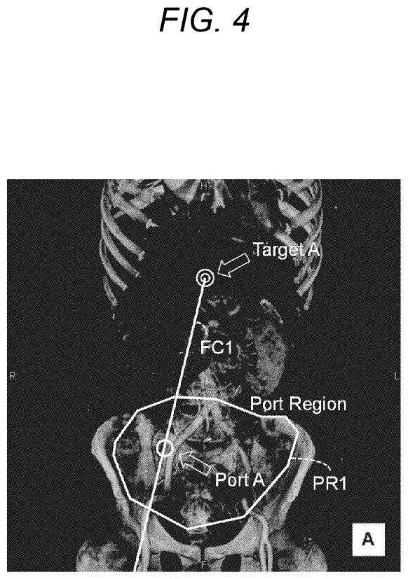

FIG. 3 is a view illustrating a first display example of the port range information. FIG. 4 is a view illustrating a second display example of the port range information. In FIG. 3, a rendering image in which the body surface is semi-transparent and port range information are displayed. In FIG. 4, a rendering image in which the body surface is transparent and the port range information are displayed. When the position of a target A is set in a three-dimensional space inside the subject, a port placement range PR1 on the body surface is derived, and the port range information of FIGS. 3 and 4 is displayed. The port range information includes, for example, information regarding the position of the target A and the position of the port placement range PR1 on the body surface of the subject. In addition, when the position of the port A is set in the port placement range PR1, the port range information may include, for example, the position of the port A and the position of forceps FC1 connecting the target A and the port A.

Even when the body surface is displayed on the rendering image or even when the body surface is not displayed or is transparent or semi-transparent, the position of the port placement range PR1 that is displayed to overlap the rendering image may be the position on the body surface. As a result, a range of the body surface required for placing the port can be clearly grasped.

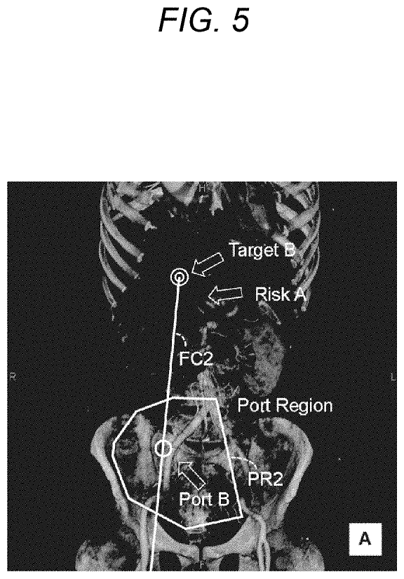

FIG. 5 is a view illustrating a third display example of the port range information. In FIG. 5, a rendering image and port range information when an obstacle A (in FIG. 5, shown as “Risk A”) is present in the subject are displayed. When the position of a target B is set in a three-dimensional space inside the subject, a port placement range PR2 on the body surface is derived based on the position of the target B and the region of the obstacle A in the subject, and the port range information of FIG. 5 is displayed. The port range information includes, for example, information regarding the position of the target B, the position of the obstacle A, and the position of the port placement range PR2 on the body surface of the subject. In addition, when the position of the port B is set in the port placement range PR2, the port range information may include, for example, the position of the port B and the position of forceps FC2 connecting the target B and the port B.

By checking the port range information illustrated in FIG. 5, the operator can insert the forceps FC2 into the set target B while avoiding interference with tissue (for example, a blood vessel, an easily damaged organ, or a bone) as the obstacle A. Accordingly, the operator can be inhibited from abruptly stabbing compressing, or pinching the tissue as the obstacle A with the forceps FC2 to damage the tissue.

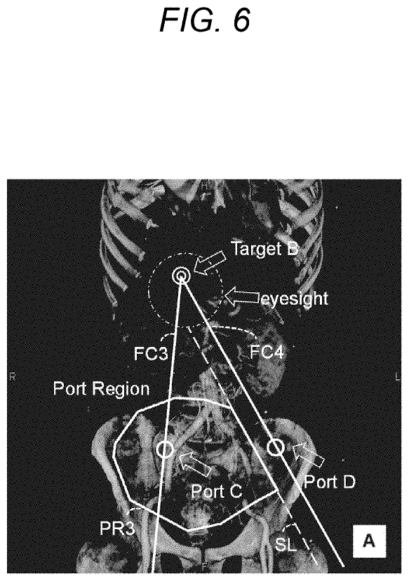

FIG. 6 is a view illustrating a fourth display example of the port range information. In FIG. 6, a rendering image in which the presence of a plurality of forceps is considered and port range information are displayed. In FIG. 6, it is assumed that forceps FC3 and FC4 inserted through a plurality of ports C and D perform treatments on the same target B.

The range information processing unit 165 calculates a port placement range PR3 of a port C of the body surface into which the forceps FC3 are inserted while avoiding a movable range of the forceps FC4. In this case, the range information processing unit 165 may calculate a movable range of the forceps FC3 with the target B as a base, and may calculate the port placement range PR3 of the port C such that the movable range of the forceps FC3 and the movable range of the forceps FC4 do not overlap each other. The movable ranges of the forceps FC3 and FC4 may be derived based on the surgical procedure in which the forceps FC3 and FC4 are used and information regarding characteristics of the forceps FC3 and FC4. The range information processing unit 165 may acquire information regarding the movable ranges of the forceps FC3 and FC4 from the memory 150, may acquire via the acquisition unit 110, or may generate.

In FIG. 6, an outer edge of the movable range of the forceps FC4 is indicated by a straight line SL at a predetermined distance (safety distance) from the forceps FC4. Therefore, a part of the outer edge of the port placement range PR3 has a shape that is cut along the straight line SL. The safety distance may be a predetermined value or a variable value. The safety distance may be determined depending on the surgical procedure or the use of the forceps. In FIG. 6, “Eyesight” represents a range (eyesight) that can be imaged by a camera as a surgical instrument inserted through an endoscopic port different from the ports C and D. In the range of the eyesight, the user can observe an internal state of the subject using acquired images during surgery. Damages of tissue caused by interference between the forceps FC3 and the forceps FC4 outside the eyesight or unexpected pinching between the forceps FC3 and the forceps FC4 outside the eyesight can be inhibited.

When the position of the target B is set in the three-dimensional space inside the subject, the port placement range on the body surface may be derived and displayed. This port placement range does not consider interference between a plurality of forceps, is wider than the port placement range PR3 of FIG. 6, and approaches, for example, the shape of the port placement range PR1 illustrated in FIGS. 3 and 4 (not illustrated in FIG. 6). It is assumed that the position of the port C is set after setting the position of the port D in the state. When the position of the port C is set after setting the position of the port D, the port placement range is recalculated based on the movable range of the forceps FC4 different from the forceps FC3 to be inserted into the port C, and the port placement range PR3 is obtained. When the position of the port C is set, the port range information includes, for example, the position of the target B, the position of the port D, the position of the forceps FC4 connecting the target B and the port D, the position of the port placement range PR3, the position of the eyesight that can be recognized by the user, and the straight line SL indicating the safety distance from the forceps FC4 outside the eyesight. When the position of the port C is set, the port range information may include, for example, the position of the port C and the position of forceps FC3 connecting the target B and the port C.

By checking the port range information illustrated in FIG. 6, the operator can check the straight line SL indicating the safety distance and can easily secure the distance between the forceps that is being used and other forceps during surgery. Accordingly, the medical image processing apparatus 100 can avoid damages of an organ or a blood vessel caused, for example, when pinched between the forceps FC3 and FC4. In particular, an organ or a blood vessel can be inhibited from being pinched outside the eyesight of a laparoscope. Here, the port placement range PR3 is derived based on the movable range of the forceps FC4 to be inserted into the port D set in advance, the movable range of the forceps FC3 to be inserted into the newly planned port C such that the movable ranges do not overlap each other. The port placement range PR3 may be derived such that interference between the laparoscope (camera) as a surgical instrument and the forceps can be avoided.

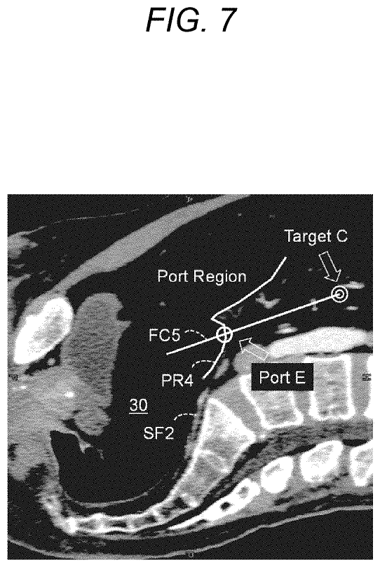

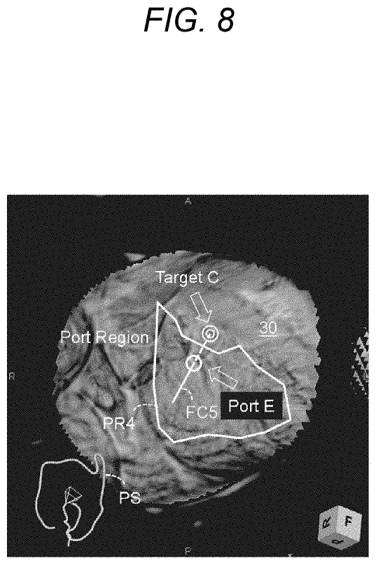

FIG. 7 is a view illustrating a fifth display example of the port range information. FIG. 8 is a view illustrating a sixth display example of the port range information. In FIGS. 7 and 8, when approaching a target from the lumen in NOTES, a rendering image and port range information are displayed. In the example of FIG. 7, the rendering image is an MPR image. In the example of FIG. 8, the rendering image is a virtual endoscopic image.

When a target C is set, the port range information illustrated in FIGS. 7 and 8 is displayed. Here, a port placement range PR4 where a port E is placeable on a luminal surface SF2 of a tubular tissue 30 (for example, rectum) is derived and displayed. The port range information illustrated in FIGS. 7 and 8 includes, for example, information regarding the position of the target C and the position of the port placement range PR4 in the luminal surface SF2 of the subject. In addition, when the position of the port E is set in the port placement range PR4, the port range information may include, for example, the position of the port E and the position of forceps FC5 connecting the target C and the port E. In the lower left end portion of FIG. 8, information PS regarding the traveling state of the tubular tissue 30 in the subject is displayed to indicate the position of the virtual endoscopic image illustrated in FIG. 8 in the tubular tissue 30.

The display control unit 166 may display an intersection between an MPR surface of an MPR image and the port placement range PR4 in the three-dimensional space of the subject to overlap the MPR image. In addition, the display control unit 166 may display an intersection between an image surface of a virtual endoscopic image in which the lumen is visualized and the port placement range PR4 in the three-dimensional space of the subject to overlap with the virtual endoscopic image. The virtual endoscopic image is an example of the three-dimensional image of the lumen. The three-dimensional image of the lumen may be a perspective projection image, a parallel projection image, or a cylindrical projection image.

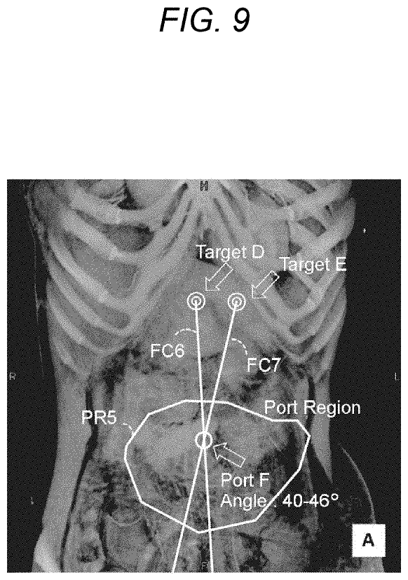

FIG. 9 is a view illustrating a seventh display example of the port range information. In FIG. 9, when a plurality of targets D and E are present, a rendering image and angle information are displayed. When the positions of the targets D and E are set, the port range information including the angle information illustrated in FIG. 9 is generated and displayed.

The port range information includes, for example, information regarding the positions of the targets D and E and the position of a port placement range PR5. In addition, when the position of a port F is set in the port placement range PR5, the port range information may include, for example, the position of the port F, the position of forceps FC6 connecting the target D and the port F, and the position of forceps FC7 connecting the target E and the port F. In addition, the angle information includes information representing that an angle AG1 between the forceps FC6 at the position of the port F and the body surface is 46° and an angle AG2 between the forceps FC7 at the position of the port F and the body surface is 40°. Therefore, in FIG. 9, the angle information includes information representing that the forceps to be inserted into the port F may be adjusted to 40° to 46°.

This way, in the medical image processing apparatus 100, when the targets D and E that are accessed by the forceps through the single port F are present, the ranges of the angles at which the forceps access the targets D and E can be displayed using the upper limit angle (for example, 46°) and the lower limit angle (for example, 40°). In FIG. 7, the two different angles depending on the directions of the forceps FC6 and FC7 are considered.

In addition, when the port placement range is derived, any position may be estimated as the position of the port F. When an angle range corresponding to the estimated position of the port F is in a predetermined safety angle range, it can be determined that the port is placeable at the estimated position of the port F. In this case, the port placement range can include the estimated position of the port.

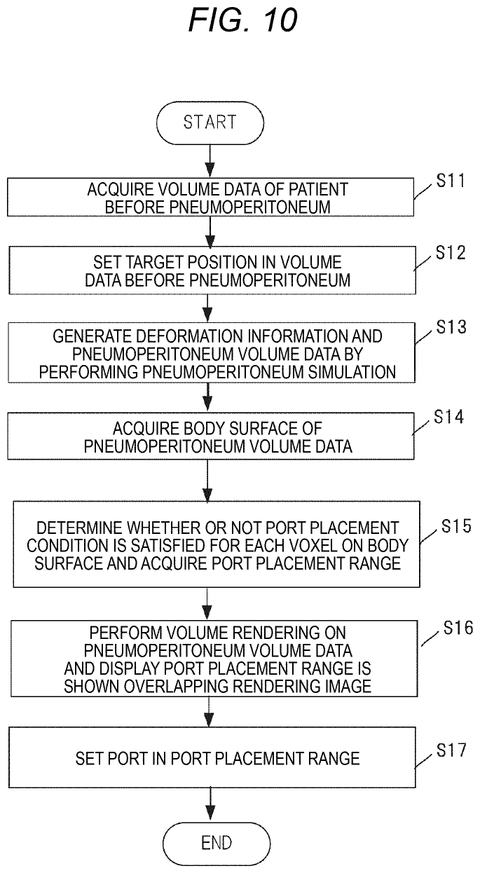

FIG. 10 is a flowchart illustrating an operation example of the medical image processing apparatus 100. In the example of FIG. 10, the port is set on the body surface of the subject.

First, volume data of a non-pneumoperitoneum state of the subject (for example, a patient) is acquired (S11). The position of a target is set in the volume data of the non-pneumoperitoneum state (S12). By performing a pneumoperitoneum simulation, volume data of a virtual pneumoperitoneum state is generated (S13). In addition, deformation information regarding a correspondence between the volume data of the non-pneumoperitoneum state and the volume data of the virtual pneumoperitoneum state is generated (S13). For example, the contour of the volume data of the virtual pneumoperitoneum state is extracted to acquire the body surface of the subject (S14). Whether or not the port placement condition is satisfied for each voxel on the body surface is determined, and the port placement range that is configured with an array of voxels satisfying the port placement condition is calculated (S15). The luminance is set and adjusted such that the body surface is transparent, volume rendering is performed on the volume data of the virtual pneumoperitoneum state, and the port placement range is shown overlapping the rendering image (S16). The port is set in the range of the port placement range (S17).

Hereinafter, various embodiments have been described with reference to the drawings. However, it is needless to say that the present disclosure is not limited to these examples. It is obvious to those skilled in the art that various changes or modifications can be conceived within the scope of the claims. Of course, it can be understood that these changes or modifications belong to the technical scope of the present disclosure.

For example, a plurality of port placement conditions may be present. For example, a plurality of safety distances for avoiding interference with an obstacle or inference with other forceps or a plurality of safety angles at which forceps are inserted may be prepared in stages. Alternatively, a plurality of port placement ranges such as a recommended port placement range or an allowable port placement range may be derived in stages.

In addition, a pneumoperitoneum simulation or actual pneumoperitoneum is not necessarily performed on the subject without performing S13 of FIG. 10. This is because pneumoperitoneum may not be performed depending on tissues (for example, the lung).

In addition, in NOTES, after the deformation processing unit 162 virtually deforms the stomach or the intestine in a deformation simulation, the range information processing unit 165 may derive the port placement range to generate the port range information and the display control unit 166 may display the port range information. This is because, for example, in endoscopic surgery, it is assumed that forceps are pressed from the lumen of tubular tissue into which an endoscope is inserted such that the lumen is pierced. In this case, for example, the port placement range of the port placed on the inner surface (luminal surface) of tubular tissue of the subject instead of the body surface of the subject may be derived.

In addition, not only in a preoperative simulation but also in intraoperative navigation, the derivation of the port placement range, the generation of the port range information, the display of the port range information, and the like may be performed. The range information processing unit 165 may store the results of the derivation of the port placement range, the generation of the port range information, the display of the port range information, and the like in the preoperative simulation in the memory 150. In the intraoperative navigation, the range information processing unit 165 may read the results of the preoperative simulation from the memory 150, and the port range information may be displayed through the display control unit 166 and the display 130.

In addition, the range information processing unit 165 may correct the read port placement range or the read port range information based on data or information obtained in the intraoperative navigation. For example, when the port placement range in the preoperative simulation and the actual port placement range are different from each other, the range information processing unit 165 may regenerate the port range information based on the actual port placement range and display this port range information. In this case, using various sensors such as a magnetic sensor, the port position, the position of the forceps, the position of the target, the position of the other forceps, the position of the obstacle, and the like may be detected. The range information processing unit 165 may calculate the port placement range in the intraoperative navigation.

In addition, endoscopic surgery to which SILS is applied may include surgery using a laparoscope, surgery using a gastroscope, surgery using a colonoscope, or the like. In addition, the above-described embodiment is applicable not only to SILS in which a camera and a plurality of forceps are inserted through one port but also to laparoscopic surgery in which a camera and a plurality of forceps are inserted through a plurality of ports.

In addition, in the above-described example, the port position is set after the deformation simulation, and the target position is set before the deformation simulation. However, the present disclosure is not limited to this example. The port position and the target position may be performed before or after the deformation simulation. The port position and the target position may be designated using a predetermined sectional image (MPR) based on the volume data of the subject, for example, via the UI 120.

In addition, the epithelial tissue in which the port is set may be the body surface of the subject or may be the luminal surface of an organ inside the subject. In addition, a laparoscope, an endoscope, forceps, an end effector of a surgical robot, and other surgical instruments will also be collectively referred to as “minimally invasive surgical instrument”.

In addition, the medical image processing apparatus 100 may include at least the processor 140 and the memory 150. The acquisition unit 110, the UI 120, and the display 130 may be externally attached to the medical image processing apparatus 100.

In addition, in the example, the volume data as the acquired CT images is transmitted from the CT scanner 200 to the medical image processing apparatus 100. Alternatively, the volume data may be stored by being transmitted to a server or the like (for example, image data server (PACS) (not shown)) on a network so as to be temporarily accumulated. In this case, the acquisition unit 110 of the medical image processing apparatus 100 may acquire volume data from the server or the like when necessary via a wired circuit or a wireless circuit or may acquire volume data via any storage medium (not shown).

In addition, in the example, the volume data as the acquired CT images is transmitted from the CT scanner 200 to the medical image processing apparatus 100 via the acquisition unit 110. This example also includes a case where the CT scanner 200 and the medical image processing apparatus 100 are integrated into one product. In addition, the example may also include a case where the medical image processing apparatus 100 is considered as a console of the CT scanner 200.

In addition, in the example, the CT scanner 200 acquires images to generate volume data including information regarding the inside of the subject. However, another device may acquire images to generate volume data. Other devices include a magnetic resonance imaging (MRI) apparatus, a positron emission tomography (PET) device, an angiography device, or other modality devices. The PET device may be used in combination with other modality devices.

In addition, the present disclosure can be implemented as a medical image processing method in which an operation of the medical image processing apparatus 100 is defined. In addition, the present disclosure can be implemented as a program for causing a computer to execute each step of the medical image processing method.

### Outline of Above Embodiment

According to one aspect of the above-described embodiment, there is provided the medical image processing apparatus 100 including the acquisition unit 110, the processing unit 160, the display unit (for example, the display 130). The acquisition unit 110 has a function of acquiring volume data of a subject. The processing unit 160 has a function of setting a target (for example, the target A) to be placed inside the subject in 3D data based on the volume data (for example, non-processed volume data of the non-pneumoperitoneum state, volume data in which the stomach or the intestine is deformed, data obtained by deforming a surface model generated from the volume data, or the volume data of the virtual pneumoperitoneum state). The processing unit 160 has a function of visualizing (for example, rendering) the 3D data and not displaying an epithelial tissue such that the display unit displays not only a transparent or semi-transparent image but also information (for example, the port range information) regarding a port placement range where a port through which a surgical instrument (for example, forceps) reaching the target is inserted is placeable on the epithelial tissue.

As a result, the medical image processing apparatus 100 can provide the information regarding the range where the port through which the surgical instrument is inserted is placeable to the user with reference to the position of the set target. Accordingly, the user can recognize the range where the port through which the surgical instrument passes is placeable on the subject, and burden on an operator and the subject can be reduced. By placing (piercing) the port in the port placement range, the user can improve the safety of a treatment on the target. Accordingly, the user can conceive a required treatment in consideration of the information regarding the port placement range in the preoperative simulation or the intraoperative navigation. In addition, by not displaying the epithelial tissue such that the epithelial tissue is transparent or semi-transparent, the user can easily grasp the port placement range, the placement of the shaft of the forceps from the port to the target, the insertion path of the forceps, and the target position intuitively at the same time while checking the target and the surrounding tissue.

In addition, the processing unit 160 may calculate the port placement range on the epithelial tissue and may set a port to be placed on the epithelial tissue of the subject in a range of the port placement range. As a result, in the medical image processing apparatus 100, by not displaying the epithelial tissue, the port placement range can be visualized in the transparent or semi-transparent epithelial tissue. Accordingly, the user can recognize the port placement range in the epithelial tissue while checking the inside of the subject.

In addition, the processing unit 160 may generate deformation information regarding deformation of the subject in a deformation simulation of the subject and may generate the 3D data based on the volume data and the deformation information. As a result, the medical image processing apparatus 100 can provide the information regarding the port placement range by calculating the port placement range corresponding to the actual state during surgery in consideration of deformation of tissue inside the subject caused by pneumoperitoneum or movement of the surgical instrument.

In addition, the target may include a plurality of points or a region in the subject. The port range information is information regarding at least one of an upper limit and a lower limit of an angle between the surgical instrument and the epithelial tissue at a position of the port. The above-described region includes a plurality of points. As a result, even when the user performs a treatment on a plurality of targets, the user can check an angle range that is adjustable by moving the surgical instrument. Accordingly, for example, when the angle range is a safety range, the user may determine that the port is placeable at an estimated position of the port.

In addition, the port placement range may be determined based on an organ on a path between the port and the target where the surgical instrument is placed. The path may be, for example, a position of a path through which the forceps inserted between the port and the target pass or may be a position of the shaft of the inserted forceps. As a result, in the medical image processing apparatus 100, for example, the position where the surgical instrument is placed and the position of an organ can be inhibited from overlapping each other, and damages of the organ can be inhibited.

In addition, at least one port may be placed. A plurality of surgical instruments may be insertable into at least one port. For example, in SILS using a single port, the degrees of freedom of movement of the surgical instruments to be inserted into the subject is low. Even in this case, when the user performs a treatment using the limited surgical instrument, the user can place the port in consideration of safety by checking the port placement range.

In addition, the surgical instruments may include a first surgical instrument and a second surgical instrument. The port placement range may be the port placement range where the first surgical instrument is inserted and is determined based on a position where the second surgical instrument is placed in the subject. As a result, in the medical image processing apparatus 100, interference with other surgical instruments can be inhibited, and the safety during surgery can be improved.

In addition, a shaft portion of the surgical instrument may have flexibility and may include a folding mechanism, and an angle of a distal end portion of the surgical instrument may be adjustable. Even in this case, the medical image processing apparatus 100 can derive the port placement range and can present the port range information in consideration of the angle range where the surgical instrument is adjusted. In addition, even when surgical instruments pass through the same port position and the same target position, in a case where the adjustment angles of the surgical instruments are different from each other, a space (working area) for performing a treatment in the subject can be easily secured.

In addition, the surgical instrument may be an end effector that is used for a surgical robot. As a result, the medical image processing apparatus 100 can support robotic surgery by presenting the port range information. In particular, in robotic surgery, the approach from the port to the target is long. Therefore, it is difficult to accurately determine the port placement position. In the medical image processing apparatus 100, when the target is designated in a preoperative simulation, a position where the port is placeable can be displayed on a visualized image.

In addition, the epithelial tissue of the subject may be a tissue of the luminal surface SF2 of the subject. The luminal surface SF2 is positioned inside the subject and thus cannot be directly recognized by the user. On the other hand, the medical image processing apparatus 100 can provide the port range information including the port placement range PR4 in the luminal surface SF2 to the user and can further improve the safety of surgery. Accordingly, the medical image processing apparatus 100 can support surgery by presenting the port range information in the preoperative simulation of NOTES or the intraoperative navigation.

According to another aspect of the above-described embodiment, there is provided a medical image processing method including: a step of acquiring volume data of a subject; a step of setting a target to be placed inside the subject in 3D data based on the volume data; and a step of visualizing the 3D data and not displaying an epithelial tissue of the subject such that a display unit displays not only a transparent or semi-transparent image but also information regarding a port placement range where a port through which a surgical instrument reaching the target is inserted is placeable on the epithelial tissue.

According to still another aspect of the embodiment, there is provided a medical image processing program for causing a computer to execute the above-described medical image processing method.

The present disclosure is useful as a medical image processing apparatus capable of recognizing a range where a port through which a surgical instrument passes is placeable on a subject and reducing burden on an operator and the subject, a medical image processing method, and a medical image processing system.

What is claimed is:

1. A medical image processing apparatus comprising:
a display unit; and
circuitry configured to:
acquire volume data of a subject;
set a target to be placed inside the subject in 3D data based on the volume data; and
visualize the 3D data to display an image and information on the display unit, wherein
in the image, an epithelial tissue of the subject is not displayed, is transparent, or is semi-transparent, and
the information represents a port placement range where a port through which a surgical instrument reaching the target is inserted is placeable on the epithelial tissue.

2. The medical image processing apparatus according to claim 1, wherein the processing unit is configured to:
calculate the port placement range on the epithelial tissue; and
set a port to be placed on the epithelial tissue of the subject in a range of the port placement range.

3. The medical image processing apparatus according to claim 1, wherein the processing unit is configured to:
generate deformation information regarding deformation of the subject in a deformation simulation of the subject; and
generate the 3D data based on the volume data and the deformation information.

4. The medical image processing apparatus according to claim 1, wherein
the target includes a plurality of points or a region in the subject, and
the information includes information regarding at least one of an upper limit and a lower limit of an angle between the surgical instrument and the epithelial tissue at a position of the port.

5. The medical image processing apparatus according to claim 1, wherein the port placement range is determined based on an organ on a path between the port where the surgical instrument is placed and the target.

6. The medical image processing apparatus according to claim 1, wherein
at least one port is placed, and
a plurality of surgical instruments are insertable into the at least one port.

7. The medical image processing apparatus according to claim 6, wherein
the plurality of surgical instruments include a first surgical instrument and a second surgical instrument, and
the port placement range is a range where the first surgical instrument is inserted and is determined based on a position where the second surgical instrument is placed in the subject.

8. The medical image processing apparatus according to claim 1, wherein
a shaft portion of the surgical instrument has flexibility or includes a folding mechanism, and
an angle of a distal end portion of the surgical instrument is adjustable.

9. The medical image processing apparatus according to claim 1, wherein the surgical instrument is an end effector that is used for robotic surgery.

10. The medical image processing apparatus according to claim 1, wherein the epithelial tissue of the subject is a tissue of a luminal surface of the subject.

11. A medical image processing method comprising:
acquiring volume data of a subject;
setting a target to be placed inside the subject in 3D data based on the volume data; and
visualizing the 3D data to display an image and information on the display unit, wherein
in the image, an epithelial tissue of the subject is not displayed, is transparent, or is semi-transparent, and
the information represents a port placement range where a port through which a surgical instrument reaching the target is inserted is placeable on the epithelial tissue.

12. A medical image processing system comprising:
a display unit; and
circuitry configured to:
acquire volume data of a subject from a CT (Computed Tomography) scanner;
set a target to be placed inside the subject in 3D data based on the volume data; and
visualize the 3D data to display an image and information on the display unit, wherein
in the image, an epithelial tissue of the subject is not displayed, is transparent, or is semi-transparent, and
the information represents a port placement range where a port through which a surgical instrument reaching the target is inserted is placeable on the epithelial tissue.

13. The medical image processing apparatus according to claim 2, wherein the processing unit is configured to:
generate deformation information regarding deformation of the subject in a deformation simulation of the subject; and
generate the 3D data based on the volume data and the deformation information.
